(19) United States
(12) Patent Application Publication
(10) Pub. No.: US 2005/0175979 A1
(43) Pub. Date: Aug. 11, 2005

(54) Methods for absolute cell counting

(76) Inventor: Paul Eirian Williams, Cardiff (GB)

Correspondence Address: KING & SCHICKLI, PLLC, 247 NORTH BROADWAY, LEXINGTON, KY, 40507, US

(21) Appl. No.: 10/504,036

(22) PCT Filed: Feb. 26, 2003
(86) PCT No.: PCT/GB03/00852

Related U.S. Application Data
(60) Provisional application No. 60/361,035, filed on Mar. 1, 2002.

(30) Foreign Application Priority Data
Mar. 1, 2002 (GB) 0204888.2
Mar. 1, 2002 (CA) 2,374,159

(51) Int. Cl. C12Q 1/00

(52) U.S. Cl. 435/4

(57) ABSTRACT

The invention concerns a method for determining the absolute cell count of an identifiable cell sub-population contained in a sample by introducing into an aliquot of said biological sample a pre-determined amount of a fluid reagent comprising a known concentration of fixed and labelled cells; then using flow cytometry to determine an absolute cell count of said cell sub-population.

This invention relates to methods for absolute cell counting and in particular, but not exclusively to methods for enumerating lymphocytes.

Flow cytometry is a major laboratory diagnostic method in cellular immunology. In one implementation, peripheral blood cells are incubated with various relevant monoclonal antibodies (mAbs) which bind to their corresponding target cell surface molecules. A different fluorochrome is covalently bonded to each different mAb so that, for example, CD3 molecules expressed on T-cells are labelled with a fluoresceinated (green) mAb, CD4 molecules with a different coloured fluorochrome (e.g. red) and CD8 with another (e.g. deep red). After being washed, the cell suspension flows in a stream such that cells flow in single file. The stream is intersected by laser light which excites the fluorochrome molecules. The interruption to the transmitted laser beam enables cells to be counted and their size measured. Laser light scattered by cells (side scatter at 90° is conventionally measured) indicates their internal structure/granularity. The spectrally-shifted fluorescence emanating from any mAbs bound to any cell is detected by photomultiplier tubes (PMT) after the 90° scattered light beam is appropriately optically split and filtered. The intensity of such detected light is proportional to the level of expression of the relevant cell surface molecules.

There is often a requirement for cells of a specific type to be accurately enumerated in absolute numbers per unit volume as opposed to simply determining the relative proportions of different cell populations in a sample. For CONFIRMATION COPY example, enumeration of CD4+ T-cell lymphocytes is essential in the evaluation of prognosis and therapy in patients infected with HIV (Reference (9)) and enumeration of CD34+ stem cells is important in the assessment of cancer patients receiving stem-cell transplantation (Reference (1)).

There are currently two ways of enumerating such cells. The dual-platform method calculates absolute cell numbers from the relative frequency of phenotypes (derived from flow cytometry) and the total White Blood Cell [WBC] count (derived from a haematology analyser). Unfortunately, this approach leads to great inter-laboratory variation in estimation of CD4+ lymphocyte counts (Reference (1)) because of errors inherent in WBC enumeration by haematology analysers.

The single-plafform method does not involve a haematology analyser, only a flow cytometer. The single platform method is more precise as it relies on either concomitant precise measurement of the fluid volume in which such suspended cells are enumerated, or the precise addition of fluorescent calibration particles to the sample. Here, a known amount of a fluid containing a known concentration of labelled synthetic beads is added to a known volume of the sample, and the cells of the population or populations of interest are suitably labelled in known manner with labels distinguishable from those of the synthetic beads. The number of cells of the population or populations of interest are counted, as well as the number of labelled synthetic beads passing through the cytometer in the same period. Knowing the relative numbers of cells of a given population and the synthetic beads, and the concentration of the synthetic beads (e.g. number of synthetic beads per ml), the absolute number of cells of the given population for may be determined, and similar calculations simultaneously or sequentially may be made.

Whilst a new generation of flow cytometers with precision fluidics may represent the long-term solution to problems of inter-laboratory variation, most laboratories still rely on flow cytometers that lack the ability to measure fluid volumes precisely.

Manufacturers of flow cytometers market systems of beads, protocols and software that purport to provide precise enumeration e.g. the PROCOUNT™ system from Becton Dickinson (Reference (2)) or Flow-Count Fluorospheres from Beckman Coulter. However, assays using such commercially available beads are expensive and this may be beyond the means of some laboratories. In developing countries where HIV infection is increasing rapidly in prevalence, the reagent costs for a routine CD4 count are considered by some to be prohibitive (Reference (8)). Major additional costs to enable a single-plafform absolute CD4 count to be made are thus impractical. In these circumstances, a simple economical way of obtaining accurate CD4+ counts might be helpful. Moreover we have found that cell enumeration using synthetic beads produced data with very poor reproducibility, large variation and major inaccuracy, with calculated lymphocyte counts much lower than those obtained by conventional methods. We believe this inaccuracy may be attributed to the beads segregating differentially from cells during the various staining and washing procedures involved in preparing the cells for analysis.

U.S. Pat. No. 5,084,394 relates to the combined use of calibrated fluorescent biological cells with calibrated fluorescent microbeads to compensate for different responses of different flow cytometers due to difference in the influence of the laser power on the fluorescence intensity of the calibration microbeads and the cell samples with increases in laser power. This technique is used to calibrate the flow cytometer prior to counting. There is no suggestion of introducing calibrated biological cells into a sample, nor of counting the absolute cell numbers.

U.S. Pat. No. 5,478,722 and its divisional U.S. Pat. No. 5,776,754 describe a reagent comprising a population of cells treated by preservation in a manner which does not significantly alter the cell surface proteins, but which renders them metabolically inert and free of proteolytic activity. The use of such reagents in a calibration routine is described, to adjust the control settings and alignment of the equipment prior to use. There is no suggestion of labelling the reagent and introducing it into a sample in use, nor of counting absolute cell numbers.

### SUMMARY OF THE INVENTION

There is therefore a need for an improved method of determining the absolute cell count in a biological sample to allow precise measurements to be taken and for reproducibility between different testing centres when measuring absolute cell numbers.

Accordingly, in one aspect, this invention provides a method for determining the absolute cell count of an identifiable cell sub-population contained in a sample, said method comprising the steps of:
(i) introducing into an aliquot of said biological sample a pre-determined amount of a fluid reagent comprising a known concentration of fixed and labelled cells;
(ii) using flow cytometry to determine a count of the cells of said cell sub-population, and to determine a count of said labelled cells, and
(iii) using said cell sub-population count and said labelled cell count to determine an absolute cell count of said cell sub-population.

Thus, for example, the identifiable blood cell population could be CD3+, CD3+/CD4+, or CD3+/CD8+ cells in a sample of peripheral blood, or indeed any of the other blood cell populations identifiable in a sample.

The fluid reagent comprises a known concentration of fixed and labelled cells. The term “labelled” is used broadly to include attachment of a label or staining or imparting any other marker characteristic to the cell to allow it to be identified and counted in a flow cytometer.

It will be appreciated that this method may be used to determine an absolute cell count of a plurality of different identifiable blood cell populations contained in a sample, by ensuring that the characteristics of each blood cell population may be separately gated on the flow cytometer.

The fixed labelled cells may be mammalian cells and more particularly mammalian leucocytes.

Where there is a ready supply, the fixed labelled cells may be human cells but we have also found that fixed labelled pig cells provide good results.

In order to provide high reproducibility, the fixed labelled cells may be obtained from a cell line such as K562, ST, Jurkats, or U937.

The fixed and labelled cells are preferably labelled with a nucleic acid dye which intercalates with the nucleic acid in the cell, but other suitable dye labelling via a different mechanism may be used. A suitable dye is Propidium iodide. The fixed and labelled cells may be fixed using a fixing agent such as paraformaldehyde.

If required the cells may be treated to at least partially inhibit or disable the cell surface proteins thereon.

Depending on the particular tests involved, it may be that the fixed and labelled cells are introduced into the sample at an early stage, with the sample subsequently undergoing e.g. culture, washing, centrifugation, etc. prior to or intermediate to one or more flow cytometry steps. Depending on the nature of these prior or intermediate processes, the fixed and labelled cells may be selected so as to have minimum interference with the other substances or processes occurring in the biological sample, or they may be selected so as actively to interfere therewith.

Most advantageously, the method of the invention can be practised using a biological sample that has been cultured. In this embodiment of the invention, cultured cells, or a suspension thereof, can be mixed with a known concentration of fixed and labelled cells, as described herein, with a view to determining the absolute cell count of an identifiable cell sub-population contained in said sample.

This particular aspect or embodiment of the invention is favoured in instances where biological samples may have to be taken at a site remote from a flow cytometry measuring device. Thus, for instance, in remote regions, for example Africa, where one wanted to screen an individual, or masses of people, biological samples could be taken at a location, thereafter cultured, and then transported to a remote site where the method of the invention could be practised. The ability to work the invention in this fashion thus makes the methodology suitable for testing biological samples where there has to be a delay between actually obtaining the sample and performing the methodology described herein.

In another aspect, this invention provides a fluid preparation for use in a method in accordance with any of the preceding claims, comprising fixed and labelled cells at a predetermined concentration.

The preparation may be sold as a standard reagent for use with a flow cytometer. Naturally, the CellBeads may also be sold in concentrated or dried form to enable the user to reconstitute a fluid preparation of the required concentration.

Whilst the invention has been described above, it extends to any inventive combination of the features set out above or in the following description.

The features and advantages of the present invention will be more clearly understood by reference to the following description and examples, which are not to be construed as limiting the invention.

### BRIEF DESCRIPTION OF THE DRAWINGS

FIG. 1 illustrates the stability of CellBeads evaluated by replicate counts using 5 dual-chamber haemocytometers.

FIG. 2 is a bivariant dot-plot showing sidescatter and FL3 profiles for CellBeads stained with propidium iodide and patent leucocytes stained with anti-CD45-PE-Cy5.

FIG. 3 is a comparison of enumeration of absolute lymphocyte counts by three methods:
(a) Lymphocyte count derived using CellBeads (i.e. a single platform flow cytometer calibrated by added CellBeads);
(b) Haematology's lymphocyte count (i.e. the absolute lymphocyte count from a haematology analyser);
(c) Conventional FACScan absolute lymphocyte count (i.e. using the haematology analyser total WBC and flow cytometer differential lymphocyte count in a dual platform method). Each pair is compared by correlation and Altman & Bland plot.

FIG. 4 is a comparison of absolute CD4+, CD8+ and CD3+ counts obtained using the two-platform procedure with a haematology WBC and flow cytometer differential vs a one-platform procedure with CellBead calibration. Regressions and Bland-Altman plots are shown.

FIG. 5 is a precision profile for absolute CD4+ lymphocyte counts evaluated by the CellBead technique.

FIG. 6 is a flow cytometer enumeration of PigBeads stained with propidium iodide.

FIG. 7 is a flow cytometer analysis of a Jurkat cell stained with propidium iodide.

FIG. 8 is a flow cytometer enumeration of lymphocytes in a patient sample using Jurkat CloneBeads stained with propidium iodide.

FIG. 9 is a flow cytometer analysis of U937 cells stained with propidium iodide.

FIG. 10 is a flow cytometer enumeration of lymphocytes in a patient sample using U937 CloneBeads stained with propidium iodide.

FIG. 11 is a flow cytometer enumeration of lymphocytes in a patient sample using PigBeads stained with propidium iodide.

FIG. 12 comprises two graphs illustrating the effect of CellBeads on the in-vitro growth pattern of human derived cell lines Jurkat and U937.

FIG. 13 illustrates the effect of CellBeads on the in-vitro growth pattern of human derived cell lines Jurkat and U937 in terms of the proportion of CellBeads in culture.

### DESCRIPTION OF THE PREFERRED EMBODIMENTS

In the Examples of the invention to be described below, an absolute cell count is made on a single platform, i.e. a flow cytometer, by introducing into a known amount of blood sample a known amount of a marker preparation of fixed, labelled or stained, leucocytes of known concentration (referred to as “CellBeads”). The blood sample and the marker preparation are mixed to ensure substantially uniform distribution, and then the mixture is run through a flow cytometer, and a count taken of the CellBeads passing the laser during a given period, together with a count of one or more populations of distinguishable cells in the mixture (e.g. by differentally labelling the different cell populations or using their different forward and side scatter properties). Then, knowing the relative counts of the CellBeads and each of the cell populations of interest, the concentration of CellBeads in the marker preparation, and the proportion of the volume of the marker preparation to that of the blood sample to which it is mixed, the concentration per volume of each of the cell populations of interest may be determined, and from that the absolute count for each.

In the first Example, the CellBeads are obtained by simultaneously staining and fixing normal human leucocytes with a propidium iodide/paraformaldehyde solution. Unlike synthetic beads, the human CellBeads behaved similarly to normal cells during cell lysis and cell-washing procedures. When known number of CellBeads were added to whole blood samples, and the numbers of CellBeads and lymphocytes determined, highly reproducible and accurate enumerations were obtained which were far more so than when using synthetic beads.

### EXAMPLE 1

Preparation of Fixed and Labelled Leucocytes (“CellBeads”)

Whole blood [16 mL] was collected from a normal volunteer into four 5 mL Vacutainers containing EDTA anticoagulant. Aliquots [1 mL] were added to four sterile 25 mL plastic screwcap tubes (Sarstedt Ltd, Leicester, U.K.), containing 20 ml of lysing solution. The contents were mixed, then left for 10 min at room temperature.

Following centrifugation at 300 g for 5 min, the supematants were discarded. The peripheral blood mononuclear cell (PBMC) pellets were then resuspended and combined into one tube, to which further PBS [15 mL] was added. This process was repeated a further three times.

All the PBMC aliquots were then pooled, before being redivided into four tubes. These were again centrifuged at 300 g for 5 min and the supernatants discarded. The cells in each tube were resuspended in freshly diluted propidium iodide dye/fixation solution [5 mL], and left at 4° C. overnight. They were then centrifuged at 300 g for 5 min, the supematant discarded, and the cells resuspended in PBS [5 mL].

All cells were then combined, and an aliquot removed, after vortexing, for counting in duplicate in a Neubauer-ruled, dual-chamber haemocytometer. The cells were then separated by centrifugation at 300 g for 5 min then resuspended in the calculated volume of 1% paraformaldehyde in PBS to give a count of approximately 1×10⁶ particles/mL. The ‘CellBeads’ were stored at 4° C. and daily counts were made on 10 aliquots. The mean of each set of 10 aliquot values were found to be stable from 72 hours onwards.

Materials

Phosphate-buffered saline (PBS) solution. Twenty tablets (Oxoid Ltd, Basingstoke) were dissolved in water (2 L). This gives sodium chloride [0.16M], potassium chloride [0.003M], sodium dihydrogen phosphate [0.008M] and potassium dihydrogen phosphate [0.001 M].

Lysing solution (Stock solution). Ammonium chloride (40.1 g), sodium bicarbonate (4.2 g) and ethylenediamine tetra-acetic acid disodium salt (1.85 g) were dissolved in water (500 mL). The stock solution was stored at 4° C. for not more than six months. Working solution was prepared daily by a 10-fold dilution in water.

1% Paraformaldehyde solution in PBS. Paraformaldehyde (1 g) was added to distilled water (90 mL) and heated in a water bath in a fume cupboard at 75° C. for 3 hours, stirring occasionally. When cool, 10 mL of concentrated PBS solution (one PBS tablet dissolved in 10 ml water) was added.

Dye Solution. Propidium iodide (P1) stock solution was prepared by dissolving P1 [20 mg] in phosphate-buffered saline (20 mL) and storing at 4° C., protected from light.

Staining/Fixation solution. Addition of 1% Tween20 solution (0.2 mL) to 1% paraformaldehyde solution (20 mL) gave a 0.01% final concentration of Tween. The addition of P1 stock solution (2 mL) gave a final concentration of 100 μg/mL of P1.

Stability of CellBeads

Counts were made, using five dual-chamber haemocytometers, on 10 aliquots of CellBeads on each day that patient samples were enumerated.

Enumeration of Cells

For all samples in which cells were enumerated, the numbers of CD3+, CD3+/CD4+, and CD3+/CD8+ cells were calculated using both the dual-platform method (using the flow cytometric differential and the haematology analyser's WBC) and the single-plafform method (involving the addition of known numbers of CellBeads to the samples).

The total WBC was obtained using an Advia [Bayer] haematology analyser. The flow cytometer used was the FACScan (Becton Dickinson) equipped with a 15 mW argon ion laser tuned to 488 nm. The FACScan has three fluorescence detection pathways whose photomultiplier tubes detect FL1 (530±30 nm, optimised for FITC), FL2 (585±42 nm, optimised for phycoerythrin {PE}) and FL3 (>650 nm, optimised for PE-Cy5). Lysys II software (Becton Dickinson) was used for data acquisition, and analysis and enumeration were performed using FlowMate (Dako) and ExCel (MicroSoft) programs.

Dual-Platform Enumeration

CD3+T-cells and the CD3+/CD4+ and CD3+CD8+ subsets were enumerated. For each blood sample, 200 μL aliquots of whole blood were stained with the appropriate dual monoclonal-antibody combinations (Sigma DUAL-TAG™, Sigma Aldrich, Poole, U.K.) CD45-FITC & CD14-PE; CD3-FITC & CD4-PE and CD3-FITC & CD8-PE by incubating in the dark for 15 min at room temperature. Then 2 mL of FACS lysing solution (Becton Dickinson) was added, the sample vortexed, and incubated for 10 min at room temperature.

The sample was then centrifuged at 300 g for 5 min, washed in 2 mL of phosphate-buffered saline, centrifuged again at 300 g for 5 min, and resuspended in 0.5 mL of 1% paraformaldehyde in PBS. They were then stored at 2-8° C. in the dark for not more than 24 hours before analysis. Lymphocytes are identified by low forward and low side scatter, with positivity to CD45 and negativity to CD14 as described by Nicholson (Reference (6)).

Single-Platform Enumeration using CellBeads (in Accordance with the Invention)

Two 100 μL aliquots of peripheral blood (PB) were stained with either CD3-FITC/CD4-PE/CD45-PE-Cy5 or CD3-FITC/CD8-PE/CD45-PE-Cy5 triple-colour monoclonal antibody combinations (Dako Ltd, Ely, U.K.) and incubated in the dark for 15 min at room temperature. Then 2 mL of FACS lysing solution (Becton Dickinson) was added, the sample vortexed and 100 μL of CellBead suspension added, the contents mixed well with the pipette tip and revortexed, and incubated for 10 min at room temperature. The sample was then centrifuged at 300 g for 5 min, washed in 2 mL of phosphate-buffered saline, centrifuged at 300 g for 5 min, and resuspended in 0.5 mL of 1% paraformaldehyde in PBS. They were then stored at 2-8° C. in the dark for not more than 24 hours before enumeration by flow cytometry.

Lymphocytes were identified by their CD45/sidescatter characteristics on the SSC/FL3 dotplot. Separate gates were set around lymphocytes and CellBeads on this dotplot and the ratio of lymphocytes to CellBeads determined from the number of events in each gate. This allowed the absolute number of lymphocytes to be calculated, knowing the volume and concentration of the CellBead suspension added initially. Selection of the gated lymphocyte population and display of CD3+/CD4+ or CD3+/CD8+ (FL1/FL2) allowed analogous calculation of the absolute CD4+ or CD8+ counts.

The precision of this procedure was assessed by taking separate duplicate aliquots from 20 patient samples through the entire procedure.

Results

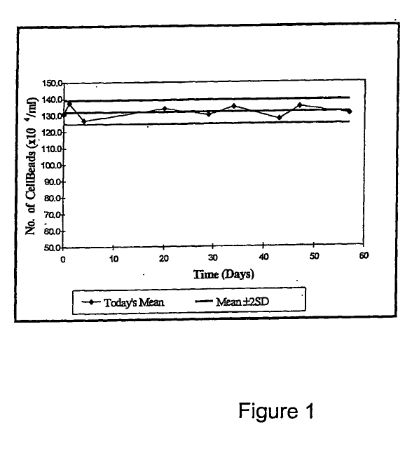

After 72 hours, CellBead counts, performed on 10 aliquots on each day that the CellBeads were used, gave constant mean cell counts and low CVs for each lot of 10 aliquots. The quality control data for one batch of CellBeads are shown in FIG. 1.

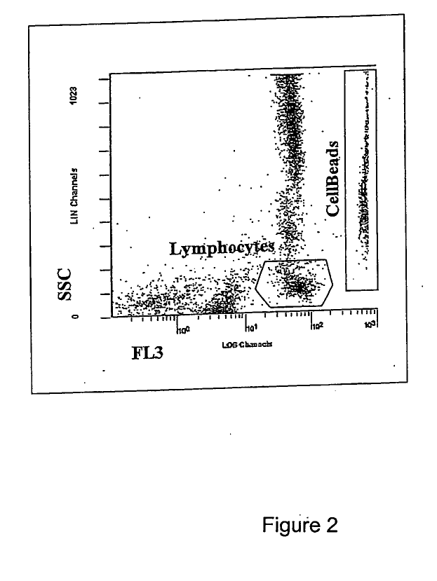

The emission spectrum of propidium iodide strongly overlaps that of phycoerythrin [PE] and of Cy5. However, the CellBeads can be clearly distinguished from the cells to be enumerated by their forward-scatter and side-scatter characteristics. FIG. 2 shows the clear distinction achieved between patient cells stained with an anti-CD45-PE-Cy5 conjugate and CellBeads stained with propidium iodide. In spite of the wash-step, some cellular debris is still apparent but this does not interfere in enumeration of cells since it has low sidescatter (SSC) and FL3 values.

The left three panels of FIG. 3 show the correlations obtained between individual lymphocyte counts determined using three methods. The methods used were:
(a) the single platform method using CellBeads to enumerate lymphocytes characterised by CD45/SSC characteristics;
(b) the dual-platform method; the first using the total WBC from a haematology analyser and the white-cell differential from the flow cytometer; and
(c) the total lymphocyte count obtained from the haematology analyser alone. It is apparent that the best agreement is between the CellBead procedure and the FACS differential procedure [coefficient of correlation of 0.9615; slope of best linear fit 1.062].

The associated Altman & Bland plots, shown in the corresponding right-hand three panels of FIG. 3, confirm satisfactory agreement between the two sets of estimates, with little indication of a tendency to increased bias at low or high lymphocyte count.

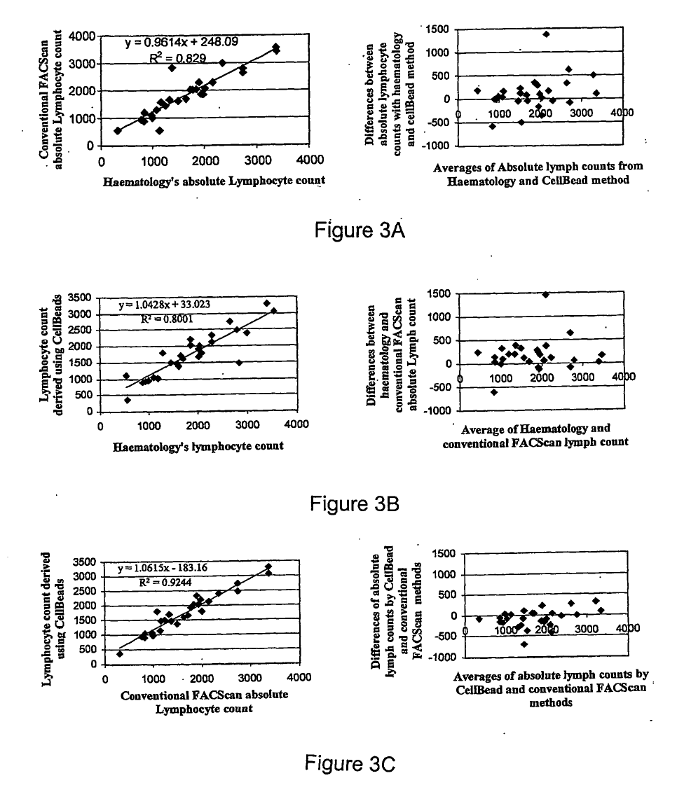

FIG. 4 presents similar data for comparison of CD3+, CD3+/CD4+ and CD3+/CD8+ numbers derived by the single-platform CellBead procedure and the dual-platform method involving the haematology analyser's total WBC and the flow cytometer's differential WBC.

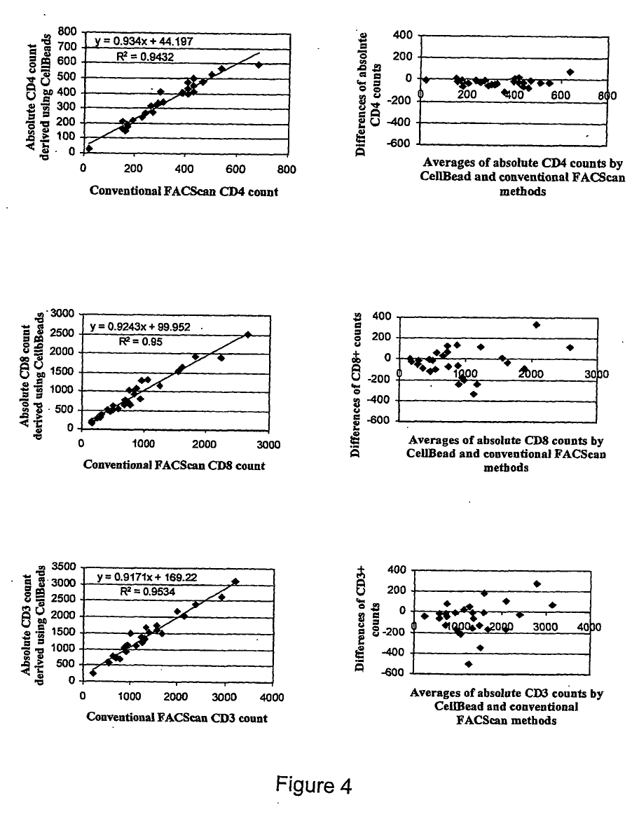

FIG. 5 shows the precision profile (plot of coefficient of variance vs mean concentration) for 20 duplicate samples processed by the single-platform procedure using CellBeads. The results demonstrate that the precision averaged (root mean square) just over 4% for CD4+ determination, and was significantly better than this (2.54%; n=6) at normal CD4+T-cell numbers (>500 million/litre).

The CellBeads in the above Example were produced from human peripheral blood leucocytes that were stained with propidium iodide and fixed in paraformaldehyde solution. Our data showed that the CellBeads were stable at 4° C. for at least two months, as indicated by the stability of the counts over time and the low C.V. when 10 aliquots were counted. Their very bright fluorescence clearly distinguished them from cells in the samples stained with anti-CD4 monoclonal antibody—fluorescent dye conjugates. This clear distinction lasted throughout the three months for which each batch was in use (data not shown).

When used for enumeration, the CellBeads produced results for lymphocyte counts (based on CD45-sidescatter gating) that were in excellent agreement with results obtained by our standard procedure (the combination of a whole blood count from a haemocytometer with the differential CD45-sidescatter proportion from the flow cytometer). There was similarly excellent agreement between absolute numbers of CD3+, CD3+/CD4+ and CD3+/CD8+ lymphocytes between the two procedures.

It has long been known that the large variation in estimates of peripheral blood CD3+/CD4+ lymphocyte numbers is due to poor reproducibility in the lymphocyte count carried out on haematology analysers (Reference (7)). Thus methods which avoid the use of haematology analysers should have superior precision (Reference (4)) if suitably calibrated by accurate volumetric (fluidics) measurements or by the addition of precise numbers of particles as internal counting standards.

Precision in the presently described method averaged just over 4% for CD4+ determinations in patients with significant CD4+ T cell lymphopenia. It was significantly better than this at normal CD4+ T cell counts. Such precision is adequate. In summary the techniques described above provides a method of counting absolute numbers of cells that is cheap, reproducible, reliable, accurate and suitable for use in any laboratory.

### EXAMPLE 2

The use of human CellBeads, that is leucocytes from a human volunteer that have been fixed with paraformaldehyde and stained with propidium iodide (PI), in the enumeration of lymphocytes or selected lymphocyte classes in patient samples provide excellent results as noted above. Ethical and practical constraints may well limit the amount of blood that can be taken from a volunteer, and other sources of suitable cells for Bead production have been explored.

PigBeads

Propidium iodide binds by intercalation to double-stranded DNA and its fluorescence is significantly increased on binding. In principle, Beads can be produced using any intercalating dye or other suitable label and leucocytes or cell lines derived from any animal species.

The pig was selected for proof in principle, since blood is readily available commercially from licensed suppliers in large (>100 mL) volumes. Fixed and labelled pig leucocytes “PigBeads” were produced essentially as described for human CellBeads in Example 1 above. Blood was processed in batches of 16 mL since this was convenient.

Leucocytes from larger volumes require a modified technique: the leucocytes produced after red cell haemolysis require gentle agitation during the staining process e.g. on a roller mixer, to ensure uniform staining. Processing larger volumes rather than numerous small batches ensures that the blood processed is fresher and improves the quality of the beads i.e. variation in sidescatter and take up of propidium iodide—as reflected in FL3 intensity on the flow cytometer, is reduced.

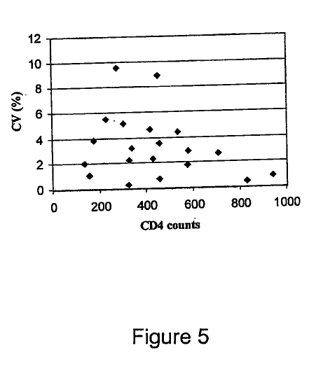

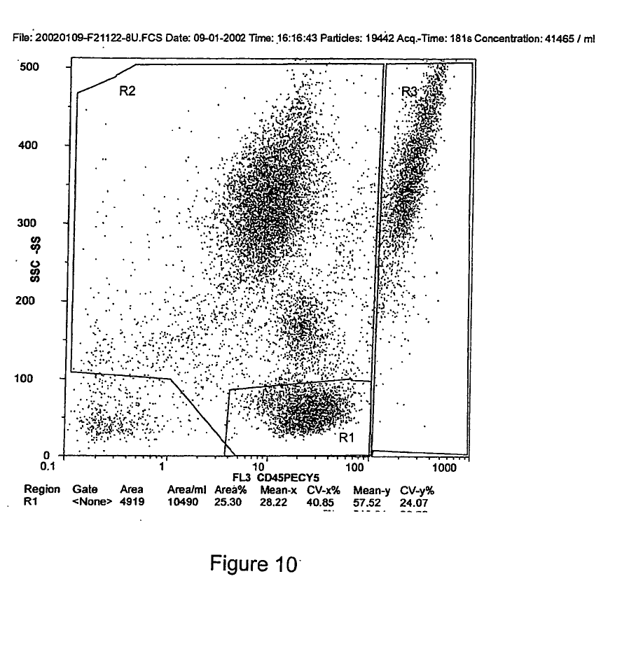

The PigBeads produced demonstrated essentially monophasic intensity in the fourth decade of FL3 (FIG. 6) and could be distinguished clearly from lymphocytes in samples from patients in biphasic plots of SideScatter vs FL3 fluorescence intensity (FIG. 11). Both human CellBeads and PigBeads were used to enumerate lymphocytes in 74 samples. Each sample was evaluated in two separate three-colour estimates using antiserum conjugates to CD3,4,45 then CD3,8,45. Lymphocytes were enumerated separately by CellBeads and by PigBeads, and by the laboratory's routine method (Cell differentiation or “CellDiffn” described below), by a single platform method elsewhere on a haematology full blood count analyser, and by a procedure (Galaxy) reliant on a precise estimate of sample volume provided by the flow cytometer (Galaxy model, Partec Gmbh).

Correlations for all regressions after removal of one obvious outlier for a Galaxy determination exceeded 0.98 in all cases. The slopes are shown below for regressions between the various procedures for lymphocyte determination. It is apparent that for the samples determined using the CD8 antibody conjugate, the results are almost identical for the slopes between estimates using beads or the routine laboratory procedure (CellDiffn) ie. 0.873 vs 0.875. The agreement is less good for the CD4 conjugate but the differences are minor and it is, in fact the PigBead data that are in better agreement with the CD8 data.

These results demonstrate that pig blood provides a readily available source of nucleated individual cells that can be stained and preserved as enumeration standards. Such PigBeads have provided reliable estimates of lymphocyte counts in routine samples. It is anticipated that blood from any other animal could be processed, with slight modifications to optimise staining, to provide CellBeads suitable for enumeration of cell populations using a flow cytometer.

Comparison of Lymphocyte Counts Determined using CellBeads or PigBeads and Other Procedures: Slopes of Linear Regressions

| CONJUGATES | BEAD | | SLOPE | |
|---|---|---|---|---|
| 3, 4, 45 | CellBeads | 1.016 | 0.954 | 1.122 |
| 3, 4, 45 | PigBeads | 0.926 | 0.862 | 1.019 |
| 3, 8, 45 | CellBeads | 0.918 | 0.873 | 1.014 |
| 3, 8, 45 | PigBeads | 0.936 | 0.875 | 1.049 |
| | | Haematol | CellDiffn | Galaxy |

Key

Haematol=Lymphocyte count determined using a single platform procedure on a haematology full blood count analyser (ADVIA).

CellDiffn=Lymphocyte count by the laboratory's current routine method using a two platform procedure. The White Blood Count from the haematology analyser is multiplied by the ratio of lymphocytes to leucocytes determined on the Galaxy flow cytometer.

Galaxy=the lymphocyte count determined by the Galaxy flow cytometer using the number of lymphocytes identified by set sidescafter and CD45 characteristics and a precise sample volume determined by the instrument. 3,8,45 refers to an analytical determination in which the leucocytes were stained with antibody conjugates to the cell differentiation markers CD3, CD8 and CD45.

### EXAMPLE 3

CloneBeads

The use of monoclonal cell-lines for production of cells which are then fixed and labelled to produce “CellBeads” has several advantages. Cultures of such cells can be expanded in vitro to produce essentially unlimited numbers of cells with uniform characteristics. Many lines are available so beads can be designed to have, or not to have, designated cellular differentiation antigens. In the event of misadventure, seed cells for well characterised lines can be obtained from national cell collections and used to restart production of cells which will have identical properties.

CloneBeads have been produced from four cell-lines: K562, ST, Jurkats and U937. Staining was modified only slightly from that described previously for CellBeads. The cell suspensions were counted and a volume equivalent to around 6×10⁷ cells, roughly equivalent to 20 mL of human blood leucocytes, was taken. The concentration of PI was reduced by 25% to 75 μg/mL and staining carried out for one hour. Such conditions optmised the intensity of Pi fluorescence in FL3 on the flow cytometer, reducing the tendency of a small number of cells to overstain and be misclassified.

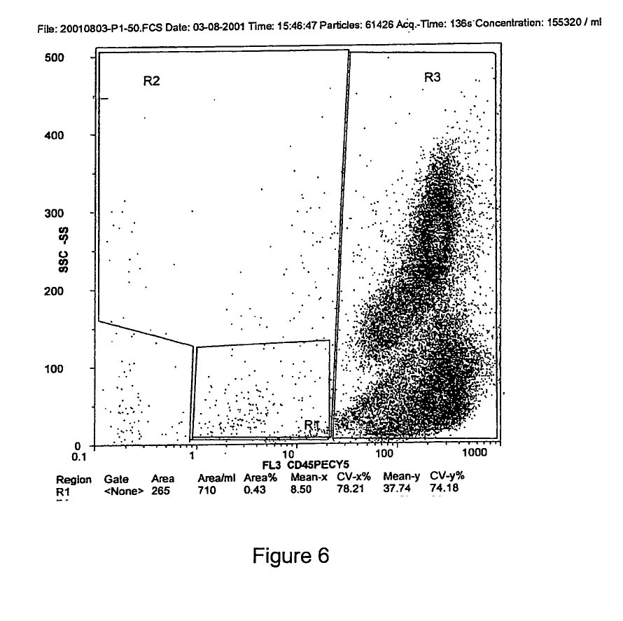

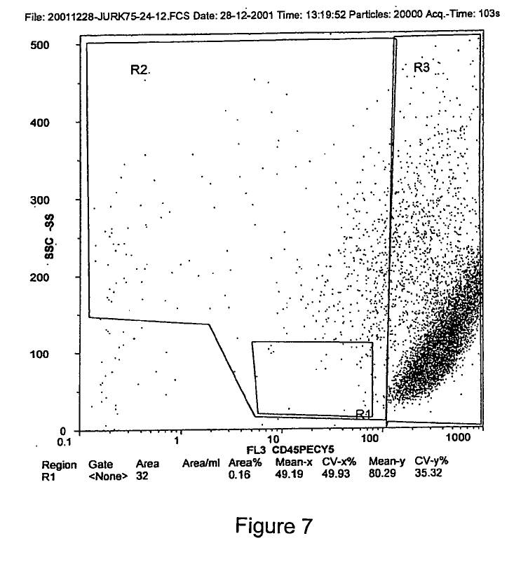

Two cell lines were selected for further evaluation. The Jurkat cells produced beads with a high intensity on FL3 that did not intrude into the lymphocyte range and a SSC largely overlapping that of lymphocytes (FIG. 7). The clear differentiation achieved from the patient sample cells is demonstrated (FIG. 8).

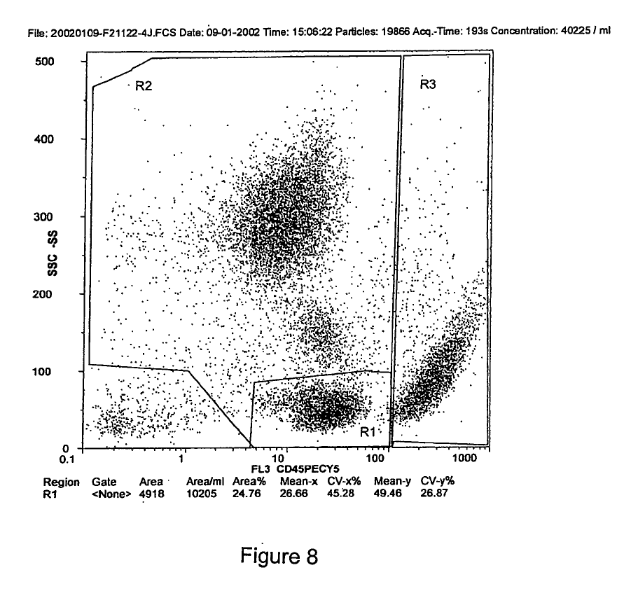

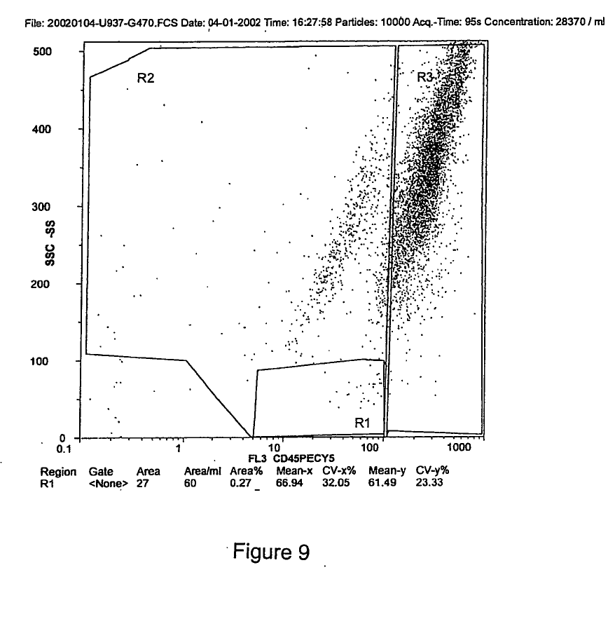

The U937 cells (a myelomonocytic line) demonstrated considerably greater sidescatter than the Jurkats or lymphocytes but the intensity of PI fluorescence in FL3 was essentially all within the fourth decade (FIG. 9). Both features combined to facilitate a very clear distinction of the CloneBeads from the sample lymphocytes (FIG. 10).

Lymphocytes from a small group of samples were enumerated using human CellBeads, PigBeads, Jurkat CloneBeads and U937 CloneBeads and the normal laboratory method, the Cell Differential procedure. Results were evaluated by normalising the bead methods by using the ratio of each enumeration to the corresponding cell differential method. All average (geometric mean) ratios were within 10% for the four bead-procedures, and in fact the two CloneBead procedures were in closer agreement with the routine procedure.

These data demonstrate that many cell lines when stained under appropriate conditions may provide CloneBeads that are well suited for cell enumeration in flow cytometer studies.

### EXAMPLE 4

We have shown previously, that cell beads can be used to accurately quantify cell number in fresh blood samples. To investigate their potential use for enumerating cells maintained in tissue culture we used two human derived cell lines Jurkats (T lymphoblastoid) and U937 (monocytic). Cell beads were prepared using cells from each of the cell lines. Cultures were established by mixing 1×10⁶ unlabelled cells with 0.2×10⁶/ml of the corresponding cell bead preparation in 10 ml culture volumes. These cultures were maintained over a 7-day period.

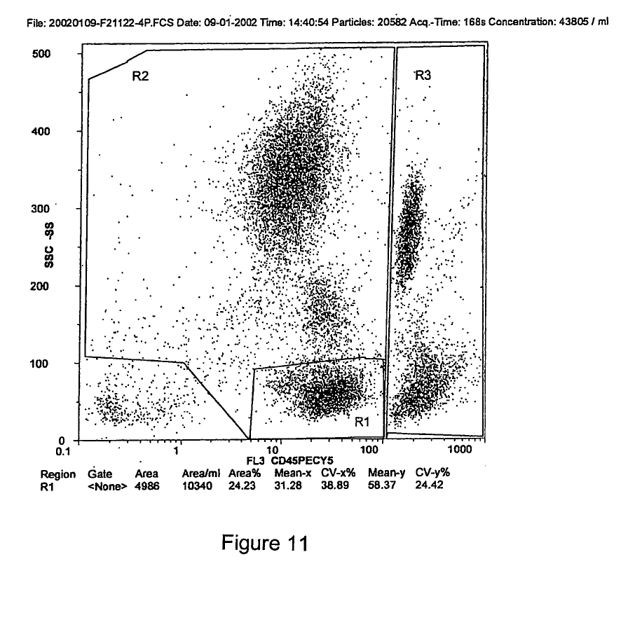

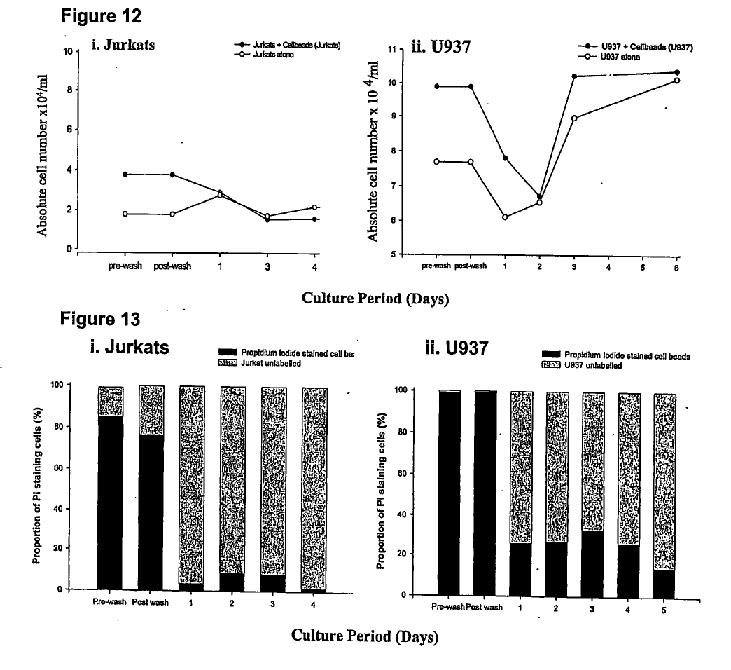

At various time points cultures were sampled and analysed for absolute cell number and proportion of cell beads. FIG. 12 shows absolute cell number obtained from each culture for Jurkats and U937. The growth pattern of each of the cell lines was not adversely affected by the presence of cell beads. The two cell lines did show differing growth patterns: Jurkats, cell numbers remaining constant and U937, in contrast, demonstrating a rapid expansion between days 2 and 3. The proportion of the cell bead population remained the same throughout the culture period (FIG. 13).

In addition, to confirm that normal immunophenotypic staining patterns were retained we determined the percentage of HLA-DR positive cells under each condition. In both culture sets, cell line alone and in combination with cell bead, the proportion of HLA-DR positive cells was the same throughout the culture period. These results demonstrate the potential use of labelled cell lines as cell beads and confirm their presence in short term cultures did not have toxic effects on other cells present in the culture.

### REFERENCES

1. Bamett D., Granger V., Whitby L., Storie I., and Reilly J T. 1999. Absolute CD4+ T-lymphocyte and CD34+stem cell counts by single-plafform flow cytometry: the way forward. Br J Haematol. 106: 1059-1062.
2. Barnett D., Bird G., Hodges E., Linch D. C., Matutes E., Newland A. C. and Reilly J. T. 1997. Guidelines for the enumeration of CD4+T lymphocytes in immunosuppressed individuals. Clin Lab Haem 19: 231-241.
3. Barbosa I. L., Sousa M. E., Godinho M. I., Sousa F., and Carvalhais A. 1999. Single-versus dual-plafform assays for human CD34+ cell enumeration. Cytometry (Communications in Clinical Cytometry) 38: 274-279.
4. Gale H. B., and Henry K. 1992. Measuring percent lymphocytes by flow cytometry to calculate absolute lymphocyte subset counts for HIV+ specimens. Cytometry 13: 175-81.
5. Lopez A., Caragol I., Candeias J. et al. 1999. Enumeration of CD4+T-cells in the peripheral blood of HIV-infected patients: an interlaboratory study of the FACSCount system. Cytometry (Communications in Clinical Cytometry) 38: 231-237.
6. Nicholson J. KA. 1994. Immunophenotyping specimens from HOV-infected persons: laboratory guidelines from the centers for disease control and prevention. Cytometry (Communicabons in Clinical Cytometry) 18: 55-59.
7. Robinson G., Morgan L., Evans M., McDermott S., Pereira S., Wansbrough-Jones M., and Griffin G. 1992. Effect of type of haematology analyser on CD4 count. Lancet 340: 485.
8. Sherman G. G., Galpin J. S., Patel J. M., Mendelow B. V. and Glencross D. K. 1999. CD4+T-cell enumeration in HIV infection with limited resources. J. Immunol. Meth. 222: 209-217.
9. Stein D. S., Korvick J. A., and Vermund S. H. 1992. CD4+Lymphocyte cell enumeration for prediction of clinical course of human immunodeficiency virus disease: a review. J. Infect. Dis. 165: 352-363.
10. Strauss K., Hannett I., Engel S., Shiba A., Ward D., Ullery S., Jinguji M. G., Valinsky J., Barneff D., Orfao A., and Kestens L. 1996. Performance evaluation of FACSCount: a dedicated system for clinical cellular analysis. Cytometry 26: 52-59.

1-33. (canceled)

34. A method for determining the absolute cell count of an identifiable cell sub-population contained in a sample, said method comprising the steps of:—
(i) introducing into an aliquot of said biological sample a pre-determined amount of a fluid reagent comprising a known concentration of fixed and labelled cells;
(ii) using flow cytometry to determine a count of the cells of said cell sub-population, and to determine a count of said labelled cells, and
(iii) using said cell sub-population count and said labelled cell count to determine an absolute cell count of said cell sub-population.

35. A method according to claim 34, wherein said fixed labelled cells are mammalian cells.

36. A method according to claim 34, wherein said fixed labelled cells are leucocytes.

37. A method according to claim 35, wherein said fixed labelled cells are human cells.

38. A method according to claim 35, wherein said fixed labelled cells are pig cells.

39. A method according to claim 34, wherein said fixed labelled cells are obtained from a cell line.

40. A method according to claim 39, wherein said fixed labelled cells are from the cell line K562.

41. A method according to claim 39, wherein said fixed labelled cells are from the cell line ST.

42. A method according to claim 39, wherein said fixed labelled cells are from the cell line Jurkats.

43. A method according to claim 39, wherein said fixed labelled cells are from the cell line U937.

44. A method according to claim 34, wherein said fixed and labelled cells are labelled with a nucleic acid dye.

45. A method according to claim 44, wherein said nucleic acid dye is Propidium iodide.

46. A method according to claim 34, wherein the fixed and labelled cells have been fixed using paraformaldehyde.

47. A method according to claim 34, wherein the fixed and labelled cells have been treated to at least partially inhibit or disable the cell surface proteins thereon.

48. A method according to claim 34, wherein step (i) is performed prior to undertaking at least one other step before performing step (ii).

49. A method according to claim 48, wherein said other step involves culturing and/or washing and/or centrifuging said biological sample and said introduced fixed and labelled cells.

50. A method according to claim 34, wherein said sample has been cultured prior to performing steps (i), (ii) and (iii).

51. A fluid preparation for use in a method in determining the absolute cell count of an identifiable cell sub-population contained in a sample, said fluid preparation comprising fixed and labelled cells at a predetermined concentration.

52. A fluid preparation according to claim 51, wherein said fluid preparation comprises said fixed and labelled cells in a selected suspension.

53. A fluid preparation according to claim 52, wherein said suspension comprises a standard reagent.

54. A fluid preparation according to claim 51, wherein said cells are mammalian.

55. A fluid preparation according to claim 51, wherein said fixed labelled cells are leucocytes.

56. A fluid preparation according to claim 51, wherein said fixed labelled cells are human cells.

57. A fluid preparation according to claim 51, wherein said fixed labelled cells are pig cells.

58. A fluid preparation according to claim 51, wherein said fixed labelled cells are obtained from a cell line.

59. A fluid preparation according to claim 58, wherein said fixed labelled cells are from the cell line K562.

60. A fluid preparation according to claim 58, wherein said fixed labelled cells are from the cell line ST.

61. A fluid preparation according to claim 58, wherein said fixed labelled cells are from the cell line Jurkats.

62. A fluid preparation according to claim 58, wherein said fixed labelled cells are from the cell line U937.

63. A fluid preparation according to claim 51, wherein said fixed and labelled cells are labelled with a nucleic acid dye.

64. A fluid preparation according to claim 63, wherein said nucleic acid dye is Propidium iodide.

65. A fluid preparation according to claim 51, wherein the fixed and labelled cells have been fixed using paraformaldehyde.

66. A fluid preparation according to claim 51 wherein the fixed and labelled cells have been treated to at least partially inhibit or disable the cell surface proteins thereon.
